(19) United States
(12) Patent Application Publication
(10) Pub. No.: US 2021/0275493 A1
(43) Pub. Date: Sep. 9, 2021

(54) IMMUNO-ONCOLOGY THERAPY USING ISOFLAVONE COMPOUNDS

(71) Applicant: NOXOPHARM LIMITED, Gordon (AU)

(72) Inventors: Graham KELLY, Gordon (AU); Olivier LACZKA, Gordon (AU)

(73) Assignee: NOXOPHARM LIMITED, Gordon (AU)

(21) Appl. No.: 17/324,927

(22) Filed: May 19, 2021

Related U.S. Application Data
(63) Continuation of application No. PCT/AU2020/050730, filed on Jul. 16, 2020.

(30) Foreign Application Priority Data
Jul. 17, 2019 (AU) 2019902518

(51) Int. Cl. A61K 31/353; A61K 39/395; A61P 35/00

(52) U.S. Cl. CPC A61K 31/353; A61P 35/00; A61K 39/3955

(57) ABSTRACT

A method for improving a response in an individual to immuno-oncology therapy for cancer.

This application is a continuation of International Patent Application No. PCT/AU2020/050730, filed Jul. 16, 2020, which claims priority to Australian Patent Application No. 2019902518, filed Jul. 17, 2019, the entirety of each of which is incorporated herein by reference.

### FIELD OF THE INVENTION

The invention relates to treatment of cancer and to use of immuno-oncology therapeutics, preferably checkpoint inhibitors, for treatment of same.

### BACKGROUND OF THE INVENTION

Immuno-oncology therapies and more particularly checkpoint inhibitor therapies are a relatively new form of treatment that cause or stimulate an immune response towards a tumor. The tumor-associated immune system is a critical determinant of patient survival and therapy success. Density and activity of cytotoxic lymphocytes such as γδ T cells, CD8⁺ T cells, or NK cells are associated with favourable prognosis, whereas the presence of suppressive myeloid cells such as macrophages or myeloid-derived suppressor cells is often a marker of poor prognosis. Thus, specific tumor immune profiles are desirable over others. This is not only true at baseline, but also following cancer therapy.

Checkpoint inhibitor therapy is associated with a survival benefit in certain tumors. PD-1 and PD-L1 blockade is effective in patients with pre-established CD8⁺ T cells that are inhibited by PD-1/PD-L1 interaction. Upregulating PD-L1, for example using IFN1, in combination with checkpoint inhibitors has shown promise in early clinical trials involving melanoma patients. Not only does IFN1 upregulate PD-L1, IFN1 also triggers TH1 associated anti-tumor immunity including cytotoxic T cell activity.

Prior to, and/or in addition to upregulation of immune checkpoints, other factors in the tumor microenvironment may prevent the establishment of active anti-tumor immunity. One of these is the presence of cells, mostly tumor cells, undergoing apoptotic cell death. Counterintuitively, apoptotic death of tumor cells is a frequent phenomenon. The interaction between apoptotic cells and, predominantly, innate immune cells such as macrophages prevents inflammation and induces self-tolerance under physiological conditions, which is exploited by the tumor. Apoptotic cell-dependent propagation of tumor development may rely on molecules such as phosphatidylserine exposed on apoptotic cells, which shape phagocyte responses, but also involves the release of signalling molecules from dying cells. Among molecules released from cells undergoing apoptosis is the sphingolipid sphingosine-1-phosphate (S1P).

S1P is a potent signalling molecule that regulates cell growth and survival and enables cancer progression, making it an attractive drug target. It is a ligand for a family of five S1P receptors (S1PRs) that regulate cytoskeletal rearrangements and cell movement, angiogenesis and vascular maturation, and immunity and lymphocyte trafficking. The S1PRs are expressed by several different cells, including immune cells; S1PR1, S1PR2 and S1PR3 are expressed ubiquitously, and S1PR4 and S1PR5 show tissue-specific distribution. The two receptors that are of interest in oncology are S1PR1 and S1PR4.

Upon binding to its receptor, S1P inhibits apoptosis and promotes proliferation through the induction of cell survival, migration and angiogenesis, the recruitment of immune cells and the evasion of the immune system. Studies in mice have shown lower levels of systemic S1P inhibit prostate cancer growth and lung metastasis.

Recruitment of immune cells is one of the ways a tumor uses S1P to its advantage. Within the tumor microenvironment both cancer and non-cancer cells secrete S1P to recruit circulating monocytes that can differentiate into macrophages. S1P also increases macrophage survival, binds to S1PR1 to attract additional macrophages, and stimulates tumor associated macrophage/M2 polarization leading to the secretion of both anti-inflammatory cytokines that help the tumor evade the immune system as well as proteins that support migration and angiogenesis.

Not all patients respond to checkpoint inhibitors, with some studies reporting only 25% of patients respond to checkpoint inhibition therapy, with up to 75% of patients showing no response at all. Additionally, some tumor types are not predicted to respond to checkpoint inhibitors alone.

Patients receiving ongoing checkpoint inhibitor therapy develop resistance which leads to poorer outcomes. Other studies have shown that late relapses of disease are emerging suggesting an acquired resistance of patients towards checkpoint inhibitor therapy.

Some checkpoint inhibitors cause sensitisation of the immune system, leading to organ-specific side effects, and a unique set of toxicities termed immune related events (irAEs). Treatment of irEAs often requires immunosuppressants therapy.

There is a need for improving the response in an individual undergoing checkpoint inhibitor therapy.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In a first aspect there is provided a method for improving a response in an individual to immuno-oncology therapy for cancer comprising the step of administering a compound of Formula 1 (described herein) to an individual in whom an improved response to immuno-oncology therapy is required, thereby improving a response to immuno-oncology therapy in the individual.

“A compound of Formula 1” generally refers to:

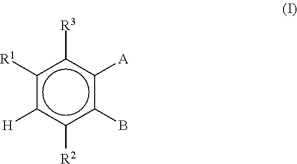

wherein

R¹ is H, C₁₋₁₀ alkyl, C₁₋₁₀ haloalkyl, OH, OR^{A} or OC(O)R^{A} where R^{A} is C₁₋₁₀ alkyl, C₁₋₁₀ haloalkyl or an amino acid;

R² is H, OH, or R^{B} where R^{B} is an amino acid or COR^{A} where R^{A} is as previously defined;

R³ is H, halo or C₁₋₁₀ alkyl.

A and B together with the atoms between them form a six membered ring selected from the group

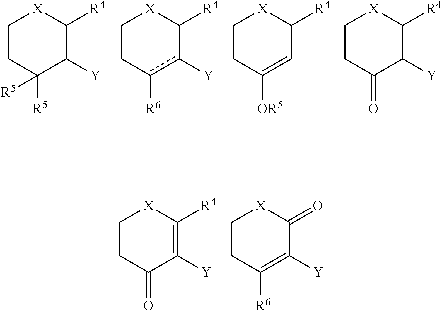

wherein

R⁴ is H, COR^{D} where R^{D} is H, OH, C₁₋₁₀ alkyl or an amino acid, CO₂R^{C} where R^{C} is C₁₋₁₀ alkyl, COR^{E} where R^{E} is H, C₁₋₁₀ alkyl or an amino acid, COOH, COR^{C} where R^{C} is as previously defined, or CONHR^{E} where R^{E} is as previously defined;

R₅ is H, CO₂R^{C} where R^{C} is as previously defined, or COR^{C}OR^{E} where R^{C} and R^{E} are as previously defined, and where the two R⁵ groups are attached to the same group they are the same or different;

R⁶ is H, CO₂R^{C} where R^{C} is as previously defined, COR^{C}OR^{E} where R^{C} and R^{E} are as previously defined, substituted or unsubstituted aryl or substituted or unsubstituted heteroaryl;

X is O, N or S;

Y is selected from the group

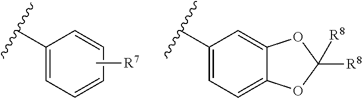

wherein

R⁷ is H, C₁₋₁₀ alkyl, C₁₋₁₀ haloalkyl, halo, OR^{F} where R^{F} is H, C₁₋₁₀ alkyl, C₁₋₁₀ haloalkyl, or OC(O)R^{A} where R^{A} is as previously defined;

R⁸ is H, halo or COR_{D} where R_{D} is as previously defined; and

“ [symbol] ” represents either a single bond or a double bond.

In a second aspect there is provided a method for conditioning an individual to improve a response in the individual to immuno-oncology therapy for cancer comprising the step of administering a compound of Formula 1 to an individual in whom conditioning to improve a response to immuno-oncology therapy is required, thereby conditioning the individual to improve a response in the individual to immuno-oncology therapy.

The method of the first or second aspect may comprise the further step of administering an immuno-oncology therapy to the individual to treat the individual for cancer.

In a third aspect there is provided a method for treating an individual for cancer comprising the step of administering a compound of Formula 1 and an immuno-oncology therapy, thereby treating the individual for cancer.

In one embodiment of the first, second or third aspect, the individual may be one who has been prior administered with immuno-oncology therapy (i.e. prior to the practice of the method of the first, second aspect or third aspect) and in whom a partial response to the immuno-oncology therapy has developed at the time that the method of the first, second or third aspect is practiced. The individual may be assessed at between 2 weeks to 52 weeks from administration of the immuno-oncology therapy prior to the practice of the method of the first, second or third aspects for a partial response to the immuno-oncology therapy. A partial response may be with respect to target or non target tumors. The method of the first, second or third aspect is to improve such a partial response. Such improvement arises from administration of the compound of Formula 1 and further administration of the immuno-oncology therapy.

In one embodiment of the first, second or third aspect, the individual may be one who has been prior administered with immuno-oncology therapy (i.e. prior to the practice of the method of the first, second aspect or third aspect) and in whom no response to the immuno-oncology therapy has developed at the time that the method of the first, second or third aspect is practiced. In this embodiment the individual may have stable disease or the individual may have progressive disease. The individual may be assessed at between 2 weeks to 52 weeks from administration of the immuno-oncology therapy prior to the practice of the method of the first, second or third aspects for stable disease. Where the individual is assessed as having stable disease at between 2 weeks to 52 weeks, the individual is administered with the compound of Formula 1 and further administered with the immuno-oncology therapy. A progressive disease may be with respect to the appearance of one or more new tumors or with respect to progression of existing target or non target tumors. Where the individual is assessed as having progressive disease at between 2 weeks and 52 weeks from administration of the immuno-oncology therapy prior to the practice of the method of the first, second or third aspects, the individual may be administered with the compound of Formula 1 and further administered with the immuno-oncology therapy. In these embodiments, the method of the first, second or third aspect is to promote a response, be it a partial response or a complete response where no response has been obtained to the immuno-oncology therapy prior to the practice of the methods of the first, second or third aspects.

In one embodiment of the first, second or third aspect, the individual has developed a partial response to immuno-oncology therapy prior to the practice of the method of the first, second or third aspect, and with the practice of the method of the first, second or third aspect, develops an improved response in the form of a complete response to immuno-oncology therapy, after the administration of a compound of Formula 1.

In another embodiment of the first, second or third aspect, the individual has developed no response to immuno-oncology therapy, more preferably, the individual has stable or progressive disease prior to the practice of the method of the first, second or third aspect, and with the practice of the method of the first, second or third aspect, develops an improved response in the form of a partial response, or a complete response to immuno-oncology therapy after the administration of a compound of Formula 1.

Thus in the above described embodiments, the individual to whom the method of the first, second or third aspects may be applied may be one who has had a partial response, or stable or progressive disease in response to the immuno-oncology therapy that has been applied prior to the practice of the method of the first, second, or third aspects.

In one embodiment of the first, second or third aspect the individual has not been administered with immuno-oncology therapy prior to the administration of a compound of Formula 1. In this embodiment, an immuno-oncology therapy may not be indicated for the particular tumor or cancer that the individual has. In this embodiment, the individual may be administered with a compound of Formula 1 and an immuno-oncology therapy according to the methods of the first, second or third aspect.

In one embodiment of the first, second or third aspect the individual has been assessed as being likely to develop a partial response, or no response to immuno-oncology therapy for cancer, prior to administration with a compound of Formula 1. Thus in one embodiment of the first, second or third aspect, the method may comprise the steps of assessing or having assessed the individual for likelihood to develop a response to immuno-oncology therapy for cancer and where an individual is assessed as having a low likelihood for development of a response to immuno-oncology therapy for cancer, administering the individual with a compound of Formula 1 and an immuno-oncology therapy.

In one embodiment of the first, second or third aspect, the individual is a patient having a solid tumor for which immuno-oncology therapy has consistently been demonstrated to be effective but for whom the immuno-oncology therapy has had limited efficacy. Such an individual may not have achieved an objective response i.e. not achieved a partial response or complete response according to RECIST 1.1) by between 2 weeks and 52 weeks following first administration of the immuno-oncology therapy prior to the practice of the methods of the first to third aspects.

In one embodiment the method of the first, second or third aspect is applicable for overcoming early treatment failure (otherwise known as primary resistance), or for managing pseudo-progression. Such an individual may demonstrate progressive disease within 2 weeks to 52 weeks following first administration of the immuno-oncology therapy prior to the practice of the methods of the first to third aspects.

In one embodiment of the first, second or third aspect, the cancer is selected from the group consisting of non-squamous non-small cell lung cancer, melanoma, renal cell carcinoma, merkel cell carcinoma, head and neck squamous cell carcinoma. In this embodiment, the individual may be one who has been given immuno-oncology therapy and who has developed a partial response to the immuno-oncology therapy prior to the practice of the method of the first, second or third aspects. In this embodiment, the individual may develop a complete response to immuno-oncology therapy.

In one embodiment of the first, second or third aspect, the cancer is selected from the group consisting of prostate cancer, pancreatic cancer, neuroblastoma, glioblastoma, sarcoma, ovarian carcinoma, Hodgkin's lymphoma, breast cancer, bladder cancer, liver cancer, colorectal cancer, oesophageal cancer, kidney cancer, skin cancer, and stomach cancer. In this embodiment, the individual may be one who has been given immuno-oncology therapy and has not responded to the immuno-oncology therapy prior to the practice of the method of the first, second or third aspects, or the individual may not have been given immuno-oncology therapy because immuno-oncology therapy is not indicated for the tumor or cancer type of the individual. In this embodiment, the individual may develop a complete or partial response to immuno-oncology therapy.

In one embodiment of the first, second or third aspect, the compound of Formula 1 is idronoxil.

In one embodiment of the first, second or third aspect, the compound of Formula 1, preferably idronoxil, is provided in the individual to establish a plasma concentration of about 40 ng/mL to about 400 μg/mLin the individual. In one embodiment of the first, second or third aspect, the compound of Formula 1, preferably idronoxil, is provided in the individual to establish a plasma concentration of about 40 ng/mL to about 400 μg/mL in the individual for a period of at least one half life of the immuno-oncology therapy.

In one embodiment of the first, second or third aspect, an immuno-oncology therapy is administered to the individual at the time that a plasma concentration of a compound of Formula 1, preferably idronoxil, of about 40 ng/mL to about 400 μg/mL has been established in the individual.

In one embodiment of the first, second or third aspect, an immuno-oncology therapy may be administered to maintain a plasma concentration as recommended by the product information pertaining to the immuno-oncology therapy for the period of time during which the plasma concentration of the compound of Formula 1, preferably idronoxil, is about 40 ng/mL to about 400 μg/m L.

In any embodiment, the plasma concentration of the compound of Formula 1 may be any concentration within the range of about 40 ng/mL to about 400 μg/mL, for example the plasma concentration may be about 40 ng/mL to about 40 μg/mL, about 40 ng/mL to about 4 μg/mL, or about 40 ng/mL to about 400 ng/mL.

In one embodiment of the first, second or third aspect, an immuno-oncology therapy and a compound of Formula 1 are administered to the individual at the same time.

In one embodiment of the first, second or third aspect, a compound of Formula 1 is administered to the individual after the administration of an immuno-oncology therapy.

In one embodiment of the first, second or third aspect, a compound of Formula 1 is administered to the individual prior to the administration of an immuno-oncology therapy.

The immuno-oncology therapy may be a checkpoint inhibitor, T-cell transfer therapy, monoclonal antibody, treatment vaccine or an immune system modulator. In any embodiment of the invention, preferably the immuno-oncology therapy is a checkpoint inhibitor therapy.

In one embodiment of the first, second or third aspect, the checkpoint inhibitor may be an immunomodulatory antibody. An immunomodulatory antibody may be a CTLA-4 inhibitor, a PD-1 inhibitor or a PD-L1 inhibitor. Preferably, the checkpoint inhibitor is a PD-1 inhibitor, more preferably nivolumab.

In one embodiment of the first, second or third aspect, the individual is not treated with, or has not been treated with radiotherapy or chemotherapy for treatment of the cancer, whether prior to the practice of the methods of the first, second or third aspects, during the practice of the methods or after completion of the methods.

In a fourth aspect there is provided a method of treating an individual for cancer, comprising the step of administering a compound of Formula 1 to the individual, wherein the individual has not responded, or has partially responded to immuno-oncology therapy, or has been assessed as likely to not respond to immuno-oncology therapy; and wherein the individual is not treated with, or has not been treated with radiotherapy or chemotherapy for treatment of the cancer. In one embodiment, the method comprises the further step of administering the individual with a therapeutically effective amount of an immuno-oncology therapy for treatment of the cancer. In this embodiment, the immuno-oncology therapy administered to the individual is the same compound as the immuno-oncology therapy to which the individual has failed to respond, or has been assessed as likely to fail to respond.

In a fifth aspect there is provided a compound of Formula 1, preferably idronoxil, for use in the treatment of cancer in an individual wherein the individual has not responded, or has partially responded to immuno-oncology therapy, or has been assessed as likely to not respond to immuno-oncology therapy; and wherein the individual is not treated with, or has not been treated with radiotherapy or chemotherapy for treatment of the cancer.

In a sixth aspect there is provided a kit including a compound of Formula 1, preferably idronoxil and an immuno-oncology therapy and written instructions for use of the kit in a method of an embodiment descried above.

In a seventh aspect there is provided the use of a compound of Formula 1, preferably idronoxil, in the manufacture of a medicament for the treatment of cancer in an individual wherein the individual has not responded, or has partially responded to immuno-oncology therapy, or has been assessed as likely to not respond to immuno-oncology therapy; and wherein the individual is not treated with, or has not been treated with radiotherapy or chemotherapy for treatment of the cancer.

In an eighth aspect there is provided a pharmaceutical composition comprising a compound of Formula 1, preferably idronoxil, or pharmaceutically acceptable salt thereof for use in the treatment of cancer in an individual wherein the individual has not responded, or has partially responded to immuno-oncology therapy, or has been assessed as likely to not respond to immuno-oncology therapy; and wherein the individual is not treated with, or has not been treated with radiotherapy or chemotherapy for treatment of the cancer.

Further aspects of the present invention and further embodiments of the aspects described in the preceding paragraphs will become apparent from the following description, given by way of example.

### BRIEF DESCRIPTION OF THE FIGURES

FIG. 1. Schematic representation of the protocol used for the coculture of C17/NPC43+ve spheroids and PBMCs from healthy donors and subsequent imaging and flow cytometric analysis.

FIG. 2. Flow cytometry analyses of T cells (respectively gated CD3⁺) as well as CD4⁺, CD8⁺ an double-positive T cells subsets (respectively gated CD4⁺ CD8⁻, CD4⁻CD8⁺ and CD4⁺ CD8⁺ among CD3⁺) percentages in the IN and OUT compartments, with or without idronoxil at 72 hours.

FIG. 3. Quantitation of flow cytometric data on the percentage of PD1⁺ and PD1⁻ memory T cells and naïve T cells in the presence of idronoxil relative to control (DMSO). *p≤0.05, **p<0.01 when compared to DMSO control.

FIG. 4. Spheroid morphology by microscopy and cell number (tumor cells and immune cell subsets) of MCF-7 (mammary carcinoma) cells after 3 days of treatment with DMSO, 1 μm idronoxil or 10 μm idronoxil, with 3 biological replicates shown, with 8 individual spheroids per group.

FIG. 5. Flow cytometry analysis (FACS) of PDL1/PD1 expression of MCF-7 spheroids after 3 days of treatments

FIG. 6. Spheroid morphology by microscopy and cell number (tumor cells and immune cell subsets) of MCF-7 cells after 6 days of treatment with DMSO, 1 μm idronoxil or 10 μm idronoxil showing IgG and anti-PD1, with 3 biological replicates shown, with 8 individual spheroids per group.

FIG. 7. Flow cytometry analysis (FACS) of PDL1/PD1 expression of MCF-7 spheroids after 6 days of treatments

FIG. 8. Spheroid morphology by microscopy and cell number (tumor cells and immune cell subsets) of A549 cells after 3 days of treatment with DMSO, 1 μm idronoxil or 10 μm idronoxil, with 3 biological replicates shown, with 8 individual spheroids per group.

FIG. 9. Flow cytometry analysis (FACS) of PDL1/PD1 expression of A549 spheroids after 3 days of treatments

FIG. 10. Spheroid morphology by microscopy and cell number (tumor cells and immune cell subsets) of A549 cells after 6 days of treatment with DMSO, 1 μm idronoxil or 10 μm idronoxil, with 3 biological replicates shown, with 8 individual spheroids per group.

FIG. 11. Flow cytometry analysis (FACS) of PDL1/PD1 expression of A549 spheroids after 6 days of treatments

### DETAILED DESCRIPTION OF THE EMBODIMENT

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognise many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text. All of these different combinations constitute various alternative aspects of the invention.

As used herein, except where the context requires otherwise, the term “comprise” and variations of the term, such as “comprising”, “comprises” and “comprised”, are not intended to exclude further additives, components, integers or steps.

For purposes of interpreting this specification, terms used in the singular will also include the plural and vice versa. For example, “a” means one or more unless indicated otherwise.

The use of the term “about” includes and describes the value or parameter per se. For example, “about x” includes and describes “x” per se. In some embodiments, the term “about” when used in association with a measurement, or used to modify a value, a unit, a constant, or a range of values, refers to variations of ±10%. For example, “about 400” in some embodiments includes 360-440.

The terms “treatment” or “treating” of a subject includes delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the individual; slowing in the rate of degeneration or decline; making the final point of degeneration less debilitating.

A “subject” herein is preferably a human subject. It will be understood that the terms “subject” and “individual” are interchangeable in relation to an individual requiring treatment according to the present invention.

The work of the inventors leading to the invention includes the unexpected finding that idronoxil promotes immuno-oncology activity in tumor cells. Through S1P inhibition in tumor cells, idronoxil promotes infiltration of T-cells into a tumor by disabling the defences of the tumor. Idronoxil modulates PD1 and PDL1 expression on T-cells and myeloid cells, therefore being immunogenic itself. In addition to promoting infiltration of T-cells in a tumor, the inventors also identified that idronoxil promotes T-cell activation, proliferation and cytotoxicity augmenting tumor killing.

“A compound of Formula 1” generally refers to:

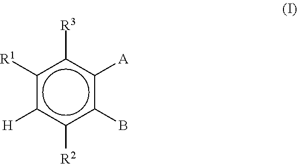

wherein

R¹ is H, C₁₋₁₀ alkyl, C₁₋₁₀ haloalkyl, OH, OR^{A} or OC(O)R^{A} where R^{A} is C₁₋₁₀ alkyl, C₁₋₁₀ haloalkyl or an amino acid;

R² is H, OH, or R^{B} where R^{B} is an amino acid or COR^{A} where R^{A} is as previously defined;

R³ is H, halo or C₁₋₁₀ alkyl.

A and B together with the atoms between them form a six membered ring selected from the group

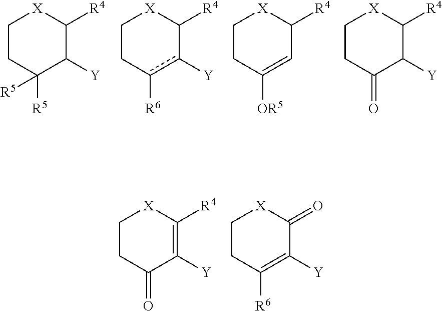

wherein

R⁴ is H, COR^{D} where R^{D} is H, OH, C₁₋₁₀ alkyl or an amino acid, CO₂R^{C} where R^{C} is C₁₋₁₀ alkyl, COR^{E} where R^{E} is H, C₁₋₁₀ alkyl or an amino acid, COOH, COR^{C} where R^{C} is as previously defined, or CONHR^{E} where R^{E} is as previously defined;

R₅ is H, CO₂R^{C} where R^{C} is as previously defined, or COR^{C}OR^{E} where R^{C} and R^{E} are as previously defined, and where the two R⁵ groups are attached to the same group they are the same or different;

R⁶ is H, CO₂R^{C} where R^{C} is as previously defined, COR^{C}OR^{E} where R^{C} and R^{E} are as previously defined, substituted or unsubstituted aryl or substituted or unsubstituted heteroaryl;

X is O, N or S;

Y is selected from the group

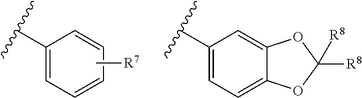

wherein

R⁷ is H, C₁₋₁₀ alkyl, C₁₋₁₀ haloalkyl, halo, OR^{F} where R^{F} is H, C₁₋₁₀ alkyl, C₁₋₁₀ haloalkyl, or OC(O)R^{A} where R^{A} is as previously defined;

R⁸ is H, halo or COR_{D} where R_{D} is as previously defined; and

“ [symbol] ” represents either a single bond or a double bond.

In preferred embodiments, the compound of formula (I) is selected from the group consisting of:

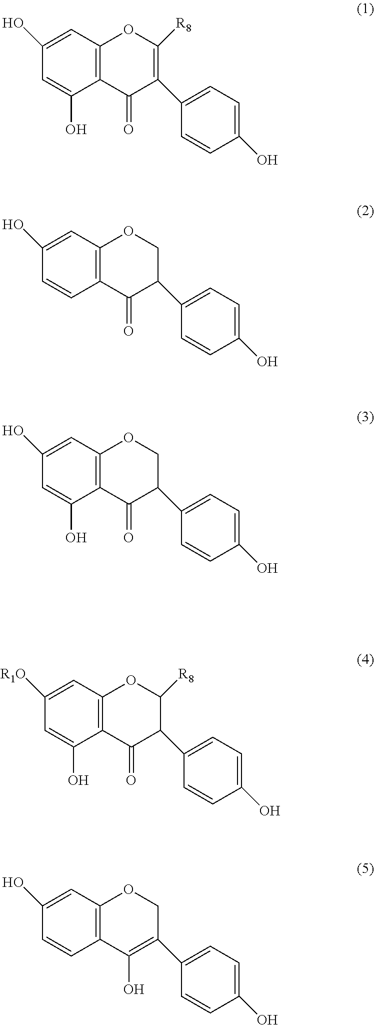

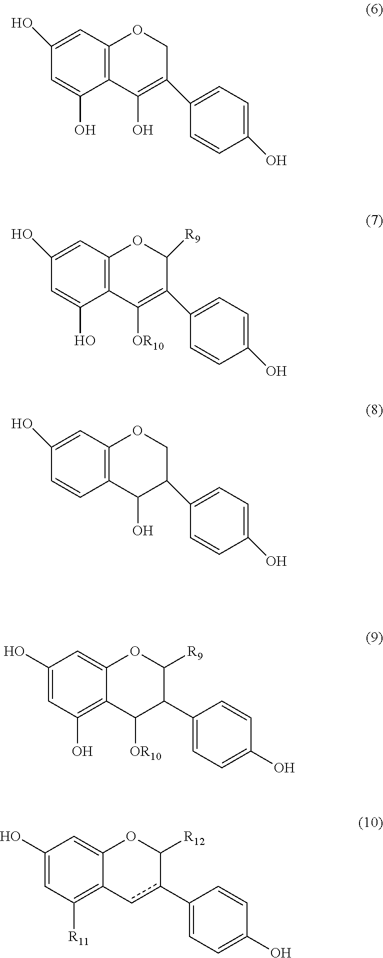

wherein

R₈ is H, halo or COR_{D} where R_{D} is as previously defined;

R₉ is CO₂R_{C} or COR_{E} where R_{C} and R_{E} are as previously defined;

R₁₀ is COR_{C} or COR_{C}OR_{E} where R_{C} and R_{E} are as previously defined;

R₁₁ is H or OH;

R₁₂ is H, COOH, CO₂R_{C} where R_{C} and is as previously defined, or CONHR_{E} where R_{E} is as previously defined; and

“ [symbol] ” represents either a single bond or a double bond.

Preferably, the compound of Formula (I) is

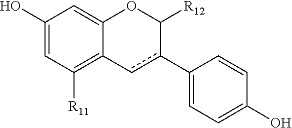

wherein R₁₁ and R₁₂ are as defined above.

Even more preferably, the compound of Formula (I) is

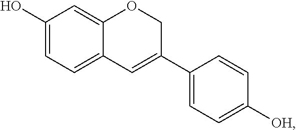 [SMILES: Oc1ccc(C2=Cc3ccc(O)cc3OC2)cc1]

otherwise known as idronoxil (also known as phenoxodiol; dehydroequol; Haginin E (2H-1-Benzopyran-7-0,1,3-(4-hydroxyphenyl)).

In another preferred embodiment, R₆ is substituted or unsubstituted aryl or substituted or unsubstituted heteroaryl. Preferably, R₆ is aryl substituted with an alkoxy group. Preferably, the alkoxy group is methoxy. In another preferred embodiment, R₆ is hydroxy.

As used herein the term “alkyl” refers to a straight or branched chain hydrocarbon radical having from one to ten carbon atoms, or any range between, i.e. it contains 1, 2, 3, 4, 5, 6, 7, 8, 9 or 10 carbon atoms. The alkyl group is optionally substituted with substituents, multiple degrees of substitution being allowed. Examples of “alkyl” as used herein include, but are not limited to, methyl, ethyl, n-propyl, isopropyl, n-butyl, isobutyl, t-butyl, n-pentyl, isopentyl, and the like.

As used herein, the term “C₁₋₁₀ alkyl” refers to an alkyl group, as defined above, containing at least 1, and at most 10 carbon atoms respectively, or any range in between (e.g. alkyl groups containing 2-5 carbon atoms are also within the range of C₁₋₁₀).

Preferably the alkyl groups contain from 1 to 5 carbons and more preferably are methyl, ethyl or propyl.

As used herein, the term “aryl” refers to an optionally substituted benzene ring. The aryl group is optionally substituted with substituents, multiple degrees of substitution being allowed.

As used herein, the term “heteroaryl” refers to a monocyclic five, six or seven membered aromatic ring containing one or more nitrogen, sulfur, and/or oxygen heteroatoms, where N-oxides and sulfur oxides and dioxides are permissible heteroatom substitutions and may be optionally substituted with up to three members. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl and substituted versions thereof.

A “substituent” as used herein, refers to a molecular moiety that is covalently bonded to an atom within a molecule of interest. For example, a “ring substituent” may be a moiety such as a halogen, alkyl group, or other substituent described herein that is covalently bonded to an atom, preferably a carbon or nitrogen atom, that is a ring member. The term “substituted,” as used herein, means that any one or more hydrogens on the designated atom is replaced with a selection from the indicated substituents, provided that the designated atom's normal valence is not exceeded, and that the substitution results in a stable compound, i.e., a compound that can be isolated, characterised and tested for biological activity.

The terms “optionally substituted” or “may be substituted” and the like, as used throughout the specification, denotes that the group may or may not be further substituted, with one or more non-hydrogen substituent groups. Suitable chemically viable substituents for a particular functional group will be apparent to those skilled in the art.

Examples of substituents include but are not limited to:

C₁-C₆ alkyl, C₁-C₆ haloalkyl, C₁-C₆ haloalkoxy, C₁-C₆ hydroxyalkyl, C₃-C₇ heterocyclyl, C₃-C₇ cycloalkyl, C₁-C₆ alkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino, acyl, carboxy, carbamoyl, aminosulfonyl, acyloxy, alkoxycarbonyl, nitro, cyano or halo.

Methods for synthesis of the above described compounds are described in WO1998/008503 and WO2005/049008 and references cited therein towards the synthesis, the contents of which are incorporated herein by reference in entirety.

Cancer immunotherapy, or immuno-oncology is the artificial stimulation of the immune system to treat cancer, improving the immune system's ability to fight the disease. The immuno-oncology therapy, or immuno-oncology therapy agent may be a checkpoint inhibitor, T-cell transfer therapy, monoclonal antibody, cancer treatment vaccine or an immune system modulator. Preferably, the immuno-oncology therapy is a checkpoint inhibitor.

A checkpoint inhibitor may be an immunomodulatory antibody. Immunomodulatory monoclonal antibody (mAb) therapies include cytotoxic T-Lymphocyte Antigen-4 (CTLA-4) inhibition (e.g., ipilimumab), Programmed Death-1 (PD-1) inhibition (e.g., nivolumab and pembrolizumab), PD-L1 inhibition, CD40 agonism, OX40 agonism, Lymphocyte Activation Gene-3 (LAG-3) and T cell Immunoglobulin Mucin-3 (TIM-3) inhibition, and Tolllike receptor agonists. Preferably, the checkpoint inhibitor is selected from: a CTLA-4 inhibitor, a PD-1 inhibitor, a PD-L1 inhibitor, or combinations thereof.

A T-cell transfer therapy may be tumor-infiltrating lymphocyte (TIL) therapy or CAR T-cell therapy. T-cell transfer therapy includes adoptive cell therapy, adoptive immunotherapy and immune cell therapy. CAR T-cell therapy includes but is not limited to Tisagenlecleucel or Axicabtagene ciloleucel.

Cancer treatment vaccines include oncolytic viruses. Cancer treatment vaccines include but are not limited to Sipulecel-T and T-VEC.

Immune modulating agents include cytokines, for example Aldesleukin, interleukins (ILs), and interferons (INFs), for example Interferon alfa-2a and/or Interferon alfa-2b, Penginterferon alfa-2b.

Immuno-mododulators, or immuno-modulatory drugs include but are not limited to inhibitors of the KIT, CSF1R and FLT3 pathways. Exemplary immunomodulatory drugs include Thalidomide, Lenalidominde, Pomalidomide, and Imiquimod.

CTLA-4 is a T cell receptor that naturally interacts with B7-1 (CD-80) and B7-2 (CD-86) on the surface of antigen presenting cells, thereby down-regulating the T cell response and avoiding potential autoimmune damage. A costimulatory T cell surface protein, CD-28, on the other hand, competes with CTLA-4, albeit with less affinity, for interaction with B7-1 and B7-2, activating the T cell. Blocking CTLA-4 thereby allows CD-28 to interact with B7-1 and B7-2, enhancing the body's cellular immune response and ability to eradicate tumor cells. For poorly immunogenic tumors, CTLA-4 blockade may be effective if used in combination with vaccination with irradiated tumor cells modified to produce GM-CSF.

PD-1 receptor is expressed on B, T, and NK cells, and interacts with Programmed Death Ligands-1 and -2 (PDL-1 and -2), often subversively expressed on melanoma cells, to induce T cell exhaustion and down-regulate the immune response. By blocking PD-1, these medications facilitate a more vigorous anti-tumor cellular immune response.

CD40 is a costimulatory receptor of the tumor necrosis factor (TNF) family normally expressed on a variety of cells including dendritic cells and macrophages. Interaction with its ligand plays a key role in priming and proliferation of antigen-specific CD4 T cells. When expressed on tumor cells, its stimulation results in apoptosis. Thus, CD40-stimulating mAbs (e.g., CD-870873) have direct anti-tumor activity and induce tumor antigen-specific T cell responses.

LAG-3 is a transmembrane protein expressed on T regulatory (T reg) cells that binds MHC II, often expressed on melanoma cells, thereby enhancing T reg activity, negatively regulating the cellular immune response, and protecting melanoma cells from apoptosis. Blocking LAG-3 could thus help the body fight tumor cells on two fronts.

Another class of immunomodulators act upon TLRs, a group of cell-surface receptors found on sentinel immune cells like dendritic cells and macrophages that naturally activate an innate immune response upon contact with characteristic pathogen-related antigens. Topical treatment of melanoma with Imiquimod (IMQ), a TLR-7 agonist, has been shown to facilitate 1) tumor infiltration with immune effector cells such as activated, cytotoxic plasmacytoid DCs, 2) a type I IFN response, 3) anti-angiogenic defenses, and in some cases result in complete tumor regression.

The blockade of TGF-β by anti-TGF-β antibody can synergistically enhance tumor vaccine efficacy, which is mediated by CD8+ T cells. For example, fresolimumab is an antibody capable of neutralizing all human isoforms of transforming growth factor beta (TGF) and has demonstrated anticancer activity.

Generating optimal “killer” CD8 T cell responses also requires T cell receptor activation plus co-stimulation, which can be provided through ligation of tumor necrosis factor receptor family members, including OX40 (CD134) and 4-IBB (CD137). OX40 is of particular interest as treatment with an activating (agonist) anti-OX40 mAb augments T cell differentiation and cytolytic function leading to enhanced anti-tumor immunity against a variety of tumors.

“Regression” and “regress” and “regresses” generally refers to the reduction in tumor size or growth of a tumor, resulting in the complete or partial involution or elimination of a tumor.

A “complete response” to therapy is generally understood as meaning the disappearance of all detectable signs of cancer in response to treatment. A complete response may arise from the elimination of tumors by immuno-oncology therapy.

A “partial response” is generally understood as meaning a decrease in tumor load in an individual, for example in terms of tumor number, size and growth rate. A partial response may increase the time to disease progression. A partial response may arise from the regression of tumors by immuno-oncology therapy.

In the embodiments of the invention described herein, a clinical response, such as a complete response or a partial response may be defined by RECIST 1.0 criteria (Therasse P, et al.) 2000 J. Natl Cancer Inst 92:2015-16 or RECIST 1.1 criteria as described in herein.

As described herein, the methods of the first to fifth aspects relate in particular to the treatment or conditioning of individual who have cancer. The methods are particularly applicable to individuals who have been prior treated with an immuno-oncology therapy and have failed that treatment in the sense that they have had only a partial response to the treatment, or they have stable or progressive disease. In these individuals the method is to applicable to condition or sensitise the individual so that subsequent treatment with immuno-oncology therapy provides an improved treatment outcome, for example, a complete response, where before the application of the methods of the first to fifth aspects only a partial response with the immuno-oncology therapy could be achieved, or where before the application of the methods of the first to fifth aspects, no response could be achieved.

In this context, it is believed that the methods of the first to fifth aspects are of particular advantage to the extent that they enable an immuno-oncology therapy to be more broadly applicable for treatment of those types of cancers, where, in the absence of these methods, immuno-oncology therapies had provided limited success. Examples of cancers to which the methods of the first to fifth aspects of the invention may be applied include blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, leukemia or lymphoid malignancies, lung cancer including small-cell lung cancer (SGLG), non-small cell lung cancer (NSGLG), adenocarcinoma of the lung and squamous carcinoma of the lung, cancer of the peritoneum, hepatocellular cancer, gastric or stomach cancer including gastrointestinal cancer, pancreatic cancer, glioblastoma, ovarian cancer, liver cancer, bladder cancer, hepatoma, breast cancer (including metastatic breast cancer), colon cancer, rectal cancer, colorectal cancer, salivary gland carcinoma, kidney or renal cancer, prostate cancer, thyroid cancer, hepatic carcinoma, anal carcinoma, penile carcinoma, testicular cancer, oesophageal cancer, tumors of the biliary tract, as well as head and neck cancer.

The individual requiring treatment may have at least two measurable tumors.

The tumors include a primary tumor.

At least one of the tumors may be a metastatic or secondary tumor of a primary tumor. The secondary cancer may be located in any organ or tissue, and particularly those organs or tissues having relatively higher hemodynamic pressures, such as lung, liver, kidney, pancreas, bowel and brain.

In one embodiment the individual has a tumor selected from the group consisting of non-squamous non-small cell lung cancer, melanoma, renal cell carcinoma, merkel cell carcinoma, head and neck squamous cell carcinoma. In this embodiment, the individual preferably has made a partial response to immuno-oncology therapy as assessed at between 2 weeks and 52 weeks from first administration of the immuno-oncology therapy and before practice of a method of the first to fifth aspects. In this embodiment, the individual is administered with a compound of Formula 1, and may further be administered with an immuno-oncology therapy.

In one embodiment, the individual may be assessed for the response to immuno-oncology therapy at about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks about 51 weeks or about 52 weeks from first administration of the immuno-oncology therapy.

In a preferred embodiment the individual preferably has made a partial response to checkpoint inhibitor therapy as assessed at between 2 weeks and 12 weeks, or any time therein, from first administration of the immune-oncology therapy and before practice of a method of the first to fifth aspects. In this embodiment, the individual is administered with a compound of Formula 1, and may further be administered with an immune-oncology therapy. The assessment of the patient and administration of the compound of Formula 1 may be made at 2 weeks, 3 weeks, 4 weeks, 5 weeks, 6 weeks, 7 weeks, 8 weeks, 9 weeks, 10 weeks, 11 weeks or 12 weeks from first administration of the immuno-oncology therapy.

In one embodiment the individual has a tumor selected from the group consisting prostate cancer, pancreatic cancer, neuroblastoma, glioblastoma, sarcoma, ovarian carcinoma, breast cancer. In this embodiment, the individual preferably has made no response to immuno-oncology therapy (i.e. has stable disease) as assessed at between 2 weeks and 52 weeks from first administration of the immuno-oncology therapy and before practice of the method of the first to fifth aspects. In this embodiment, the immune-oncology therapy is continued and the individual is administered with a compound of Formula 1. Where the individual has progressive disease in response to immuno-oncology therapy as assessed within 24 weeks from first administration of the immuno-oncology therapy and before practice of a method of the first to fifth aspects, the immuno-oncology therapy is continued and the individual is further administered with a compound of Formula 1.

Assessment of a complete or partial response, or no response to immuno-oncology therapy may be undertaken according to the methods described further herein.

Where an individual has not been prior administered with an immuno-oncology therapy prior to the practice of the methods of the first to fifth aspects, the likely response of the individual to immuno-oncology therapy can be assessed or determined prior to the administration of the compound of Formula 1 and the immuno-oncology therapy. More particularly, tumor responsiveness can be foreshadowed by investigation of tumor histological phenotype and/or immune phenotype. Selection based on immune phenotype follows a stratification of tumors into likely responsiveness to immuno-oncology therapy based on type, density and location of immune cells within the tumor site. See for example Fuereder T. 2019 MEMO 12:123-127.

Subjects requiring treatment include those already having a benign, pre-cancerous, or non-metastatic tumor as well as those in which the occurrence or recurrence of cancer is to be prevented.

The objective or outcome of treatment may be to reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder.

Efficacy of treatment can be measured by assessing the duration of survival, time to disease progression, the response rates (RR), duration of response, and/or quality of life.

In one embodiment, the method is particularly useful for delaying disease progression.

In one embodiment, the method is particularly useful for extending survival of the human, including overall survival as well as progression free survival.

In one embodiment, the method is particularly useful for providing a complete response to therapy whereby all signs of cancer in response to treatment have disappeared. This does not always mean the cancer has been cured.

In one embodiment, the method is particularly useful for providing a partial response to therapy whereby there has been a decrease in the size of one or more tumors or lesions, or in the extent of cancer in the body, in response to treatment.

“Pre-cancerous” or “pre-neoplasia” generally refers to a condition or a growth that typically precedes or develops into a cancer. A “pre-cancerous” growth may have cells that are characterized by abnormal cell cycle regulation, proliferation, or differentiation, which can be determined by markers of cell cycle.

In one embodiment, the cancer is pre-cancerous or pre-neoplastic.

“A condition or symptom associated” with the cancer may be any pathology that arises as a consequence of, preceding, or proceeding from the cancer. For example, where the cancer is a skin cancer, the condition or relevant symptom may be microbial infection. Where the cancer is a secondary tumor, the condition or symptom may relate to organ dysfunction of the relevant organ having tumor metastases. In one embodiment, the methods of treatment described herein are for the minimisation or treatment of a condition or symptom in an individual that is associated with a cancer in the individual.

In the above described embodiments, the methods according to the invention may be useful for preventing doubling time of the cancer cells or otherwise inhibiting tumor growth, either through cytotoxic effect on the tumor cells or otherwise by generally inhibiting cell replication.

In the method of the first to fifth aspects, a compound of Formula 1, preferably idronoxil may be administered at dose ranges of 400 mg daily to 2400 mg daily. For example a compound of Formula 1, preferably idronoxil may be administered at a daily dose of 400 mg, 600 mg, 800 mg, 1200 mg, 1600 mg, 1800 mg, 2000 mg or 2400 mg. In the method of the first to fifth aspects, a compound of Formula 1, preferably idronoxil may be administered in any therapeutically effective form, including but not limited to: rectal, oral, intravenous, topical, intravesical or parenteral. Preferably, the compound of Formula 1 is administered as a suppository.

In the method of the first to fifth aspects, an immuno-oncology therapy, preferably a checkpoint inhibitor, may be administered in any therapeutically effective form, including but not limited to: rectal, oral, intravenous, topical, intravesical or parenteral. Preferably, the immuno-oncology therapy is administered intravenously.

In the method of the first to fifth aspects, a compound of Formula 1, preferably idronoxil may be given for 7-14 days of every cycle, regardless of the duration of the immuno-oncology cycles (i.e. whether 2 weekly, 3 weekly or 4 weekly).

In the method of the first to fifth aspect, the sequencing of dosing of a compound of Formula 1, preferably idronoxil in each cycle and immuno-oncology may be as follows; either:
dosing of a compound of Formula 1, preferably idronoxil should precede the immuno-oncology therapy i.e. a compound of Formula 1, preferably idronoxil will be given on days 1 to 10 and the immuno-oncology compound should be given on day 2; or
dosing of a compound of Formula 1, preferably idronoxil should precede the immuno-oncology therapy i.e. a compound of Formula 1, preferably idronoxil will be given on days 1 to 7 or 14 and the immuno-oncology compound should be given on day 8; or
dosing of the immuno-oncology compound should substantially precede dosing of a compound of Formula 1, preferably idronoxil i.e. the immuno-oncology agent will be given on day 1 and a compound of Formula 1, preferably idronoxil will be given on days 8 to 14 or 17;
dosing of a compound of Formula 1, preferably idronoxil is simultaneous with the dosing of the immuno-oncology compound, i.e the immuno-oncology agent will be given on day 1 and a compound of Formula 1, preferably idronoxil, will be given on day 1.

In one embodiment wherein dosing of a compound of Formula 1, preferably idronoxil is given on days 1 to 10 and the immuno-oncology compound is given on day 2, preferably no treatment is given on days 11 to 14. In this embodiment, preferably the treatment cycle is 2 weekly.

In another embodiment wherein dosing of a compound of Formula 1, preferably idronoxil is given on days 1 to 10 and the immuno-oncology compound is given on day 2, preferably no treatment is given on days 11 to 28. In this embodiment, preferably the treatment cycle is 4 weekly.

Cycling of a compound of Formula 1, preferably idronoxil may be continued until disease progression as monotherapy, if the decision is made to stop the immune-oncology treatment (for reasons other than disease progression).

The dosage of the immuno-oncology therapy should be the dose selected by the treating physician, within the range recommended by the manufacturer for the indication.

The invention may include the further step of assessing one or more organs or tissues of an individual who has received the compound and immuno-oncology therapy, to determine the regression of a tumor in the individual. In one embodiment the step utilises radiological imaging to determine the location and volume for each of the plurality of tumor lesions in the subject after immuno-oncology therapy administration. For example, this can involve three-dimensional radiological images of the subject registering geographic locations of each of the plurality of tumor lesions. Non-limiting examples of radiological images that can be used to determine location and/or volume of a tumor lesion include positron emission tomography (PET) scans, x-ray computerized tomography (CT), magnetic resonance imaging (MRI), nuclear magnetic resonance imaging (NMRI), magnetic resonance tomography (MRT), or a combination thereof.

In one embodiment, all tumors regress.

In another embodiment, one or more tumors are eliminated.

In another embodiment, all tumors are eliminated.

In certain embodiments, the assessment of treatment follows the RECIST 1.0 or 1.1 criteria as follows:

RECIST 1.0 Criteria

Definition of Measurable and Non-Measurable Disease

Measurable disease: The presence of at least one measurable lesion.

Measurable lesion: Lesions that can be accurately measured in at least one dimension, with the longest diameter (LD) being:
≥20 mm with conventional techniques (medical photograph [skin or oral lesion], palpation, plain X-ray, CT, or MRI),

OR
≥0 mm with spiral CT scan.

Non-measurable lesion: All other lesions including lesions too small to be considered measurable (longest diameter<20 mm with conventional techniques or <10 mm with spiral CT scan) including bone lesions, leptomeningeal disease, ascites, pleural or pericardial effusions, lymphangitis cutis/pulmonis, abdominal masses not confirmed and followed by imaging techniques, cystic lesions, or disease documented by indirect evidence only (e.g., by lab values).

Methods of Measurement

Conventional CT and MRI: Minimum sized lesion should be twice the reconstruction interval. The minimum size of a baseline lesion may be 20 mm, provided the images are reconstructed contiguously at a minimum of 10 mm. MRI is preferred, and when used, lesions must be measured in the same anatomic plane by use of the same imaging sequences on subsequent examinations. Whenever possible, the same scanner should be used.

Spiral CT: Minimum size of a baseline lesion may be 10 mm, provided the images are reconstructed contiguously at 5 mm intervals. This specification applies to the tumors of the chest, abdomen, and pelvis.

Chest X-ray: Lesions on chest X-ray are acceptable as measurable lesions when they are clearly defined and surrounded by aerated lung. However, MRI is preferable.

Clinical Examination: Clinically detected lesions will only be considered measurable by RECIST criteria when they are superficial (e.g., skin nodules and palpable lymph nodes). In the case of skin lesions, documentation by color photography—including a ruler and patient study number in the field of view to estimate the size of the lesion—is required.

Baseline Documentation of Target and Non-Target Lesions

All measurable lesions up to a maximum of five lesions per organ and ten lesions in total, representative of all involved organs, should be identified as target lesions and recorded and measured at baseline.

Target lesions should be selected on the basis of their size (lesions with the LD) and their suitability for accurate repeated measurements (either clinically or by imaging techniques).

A sum of the LD for all target lesions will be calculated and reported as the baseline sum LD. The baseline sum LD will be used as a reference by which to characterize the objective tumor response.

All other lesions (or sites of disease) should be identified as non-target lesions and should also be recorded at baseline. Measurements of these lesions are not required, but the presence or absence of each should be noted throughout follow-up.

Documentation of indicator lesion(s) should include date of assessment, description of lesion site, dimensions, and type of diagnostic study used to follow lesion(s).

All measurements should be taken and recorded in metric notation, using a ruler or callipers.

Response Criteria

Disease assessments are to be performed every 6 weeks after initiating treatment. However, subjects experiencing a partial or complete response must have a confirmatory disease assessment at least 28 days later. Assessment should be performed as close to 28 days later (as scheduling allows), but no earlier than 28 days.

Definitions for assessment of response for target lesion(s) are as follows:

Evaluation of Target Lesions

Complete Response (CR)—disappearance of all target lesions.

Partial Response (PR)—at least a 30% decrease in the sum of the LD of target lesions, taking as a reference, the baseline sum LD.

Stable Disease (SD)—neither sufficient shrinkage to qualify for PR nor sufficient increase to qualify for progressive disease (PD), taking as a reference, the smallest sum LD since the treatment started. Lesions, taking as a reference, the smallest sum LD recorded since the treatment started or the appearance of one or more new lesions.

Evaluation of Non-Target Lesions

Definitions of the criteria used to determine the objective tumor response for non-target lesions are as follows:

Complete Response—the disappearance of all non-target lesions.

Incomplete Response/Stable Disease—the persistence of one or more non-target lesion(s).

Progressive Disease—the appearance of one or more new lesions and/or unequivocal progression of existing non-target lesions.

Evaluation of Overall Response for RECIST-Based Response

The overall response is the best response recorded from the start of the treatment until disease progression/recurrence is documented. In general, the subject's best response assignment will depend on the achievement of both measurement and confirmation criteria.

The following table presents the evaluation of best overall response for all possible combinations of tumor responses in target and non-target lesions with or without the appearance of new lesions.

| Target Lesion | Non-Target Lesion | New Lesion | Overall response |
|---|---|---|---|
| CR | CR | No | CR |
| CR | Incomplete response/(SD) | No | PR |
| PR | Non-PD | No | PR |
| SD | Non-PD | No | SD |
| PD | Any | Yes or No | PD |
| Any | PD | Yes or No | PD |
| Any | Any | Yes | PD |

Note:
Subjects with a global deterioration of health status requiring discontinuation of treatment without objective evidence of disease progression at that time should be classified as having ″symptomatic deterioration″. Every effort should be made to document the objective progression even after discontinuation of treatment.

In some circumstances, it may be difficult to distinguish residual disease from normal tissue. When the evaluation of complete response depends on this determination, it is recommended that the residual lesion be investigated (fine needle aspirate/biopsy) to confirm the complete response status.

Confirmation Criteria

To be assigned a status of PR or CR, a confirmatory disease assessment should be performed no less than 28 days after the criteria for response are first met.

To be assigned a status of SD, follow-up measurements must have met the SD criteria at least once after study entry at a minimum interval of 12 weeks.

A compound of Formula 1, preferably idronoxil may be formulated to form a pharmaceutical composition comprising a therapeutically effective amount of a compound of Formula 1, preferably idronoxil, and a pharmaceutically acceptable carrier that is adaptable for administration by any acceptable route. The compound of Formula 1, preferably idronoxil or pharmaceutical composition comprising same may be given orally, rectally, parenterally, by injection or other route. In one embodiment of the first to fifth aspects, the compound of Formula 1, preferably idronoxil or pharmaceutical composition comprising same is administered rectally in the form of a suppository. See for example WO2017/173474.

RECIST 1.1 Criteria

The RECIST 1.1 criteria are the same as the RECIST 1.0 criteria with updates to certain definitions recited as follows.

Number of lesions to be assessed: maximum of five total and maximum of two per organ.

Disease progression: 5 mm absolute increase additional to the definition of progression in target disease of 20% increase.

Differences in Definition of Measurable and Non-Measurable Disease

Measurable lesion: Lesions that can be accurately measured in at least one dimension, with the longest diameter (LD) being:
≥20 mm with chest X-ray,
≥0 mm with calliper measurement by clinical exam,
OR
≥10 mm with CT/MRI scan (CT scan slice thickness no greater than 5 mm or 2× slice thickness if the slice thickness is >5 mm).

Non-measurable lesion: All other lesions including lesions too small to be considered measurable (longest diameter<20 mm with chest x-ray, <10 mm with callipers or those which cannot be accurately measured with callipers or <10 mm with CT or MRI scan) including bone lesions, leptomeningeal disease, ascites, pleural or pericardial effusions, lymphangitis cutis/pulmonis, abdominal masses not confirmed and followed by imaging techniques, cystic lesions, or disease documented by indirect evidence only (e.g., by lab values).

PET may be considered to support CT, primarily for PD (detection of new lesions) or confirmation of CR.

Confirmation of partial (PR) to complete response (CR) is required only for non-randomised trials in which the ORR (objective response rate) is a key endpoint.

### BIOLOGICAL EXAMPLES

### Example 1

Spheroid Formation

For the generation of tumor spheroids in 3D, 200 μL/well of cell suspension in culture medium was seeded at cell densities of 20,000 cells/well. NPC (C17/NPC43+ve) cells were dispensed into Nunclon Sphera 96 wells plate (Thermo Fisher Scientific). The Nunclon Sphera surface was designed to cause minimal cell attachment with minimal extracellular matrix protein binding to the plate surfaces. Plates were incubated at 37° C. and 5% CO₂ in RPMI supplemented with FBS and ROCK inhibitor.

Spheroid-Infiltrating Cells Flow Cytometric Staining

For flow cytometry analyses, 8 wells/condition were seeded. OUT and IN compartments were isolated by first pooling the 8 cocultures wells in eppendorf tubes. Spheroids were gently resuspended and left to sediment to the bottom of the tubes. Supernatant cell suspension constituted the non-infiltrating immune cells (=OUT). These steps were repeated twice with Phosphate Buffer Saline (PBS) in order to separate the spheroids from the non-infiltrating immune cells. Spheroids were then trypsinized to obtain a single cell suspension (=IN) and further analyzed by flow cytometry.

Statistics

Differences in quantitative variables were analyzed by the Mann-Whitney U test when comparing 2 groups. A p value<0.05 was considered as statistically significant.

Idronoxil Treatment Increases Tumor Cell Apoptosis and Tumor-Infiltrating Cells in Nasopharyngeal Cancer Cell Line-Derived Spheroids

To mimic in vivo conditions, the cytotoxicity of idronoxil on NPC cells using three-dimensional (3D) tumor cultures was examined. The effect of increasing concentrations of idronoxil was monitored on apoptosis using fluorescent probes (non-toxic CellEvent Caspase-3/7 Green and LysoTracker Deep Red) in combination with bright-field microscopy to perform persistent real-time spheroid imaging. It was found that the intensity of C17 spheroid staining with CellEvent was proportional to idronoxil concentrations. When LysoTracker was added to the spheroids, the staining pattern was complementary to that of CellEvent, with the LysoTracker constantly accumulating in the outer and presumably metabolically active layers of the spheroids. The drug effect in the 3D “CellEvent,” was measured and used to build a dose-response curve to determine the IC₅₀ values for idronoxil at 2.1 μM. Overall, the IC₅₀ values were comparable between the spheroid-derived cells in 3D and 2D conditions.

### Example 2

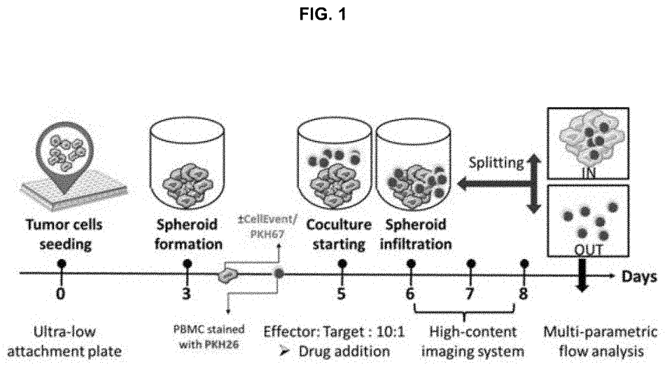

The interactions between tumor spheroids and PBMCs through heterotypic cocultures was explored. Spheroids were generated from an NPC cell line cocultured with PBMCs obtained from healthy donors that were HLA-matched whenever possible. After coculture, lymphocyte tumor infiltration and tumor cell apoptosis were measured. The cellular compositions within infiltrated tumor spheroids by mechanically separating infiltrating cells (IN) and cells remaining in the medium (OUT) were studied. A description of the coculture experimental protocol is illustrated in FIG. 1.

An increase in PBMCs infiltration in the tumor spheroids upon treatment was detected as early as 2 hours after the beginning of the experiment, while the number of infiltrated cells remained constant in the controls (DMSO) over 72 hours of coculture. It was found that the infiltration of PBMCs into tumor spheroids was enhanced over time upon idronoxil treatments. Idronoxil treatment significantly increased the influx of PBMC into the spheroids. Moreover, the CellEvent staining was performed on the coculture to confirm the induction of apoptosis and observed that spheroid infiltration was proportional to an active apoptosis process in tumor cells.

Idronoxil-conditioned tumor cells activate PBMCs that in turn, infiltrate the tumor under the influence of chemokine gradients. The in vitro exposure of NPC cell lines to idronoxil resulting in the expression of the T-cell chemokines CXCL8, 9 and 10 supports this hypothesis. Neutralizing antibodies were added to the tumor spheroid cultures. The blockade of one of the chemoattractants, CXCL10, significantly reduced idronoxil-induced PBMC migration. Collectively, these experiments demonstrate that idronoxil not only arrests tumor growth, but also induces the expression of T-cell chemoattractants, thereby enhancing T-cell infiltration, possibly augmenting further tumor killing.

### Example 3

Idronoxil administration induces differential expression of activation and homing markers in T cells infiltrates. Activated/memory T cells are able to infiltrate tumor spheroids upon idronoxil treatment.

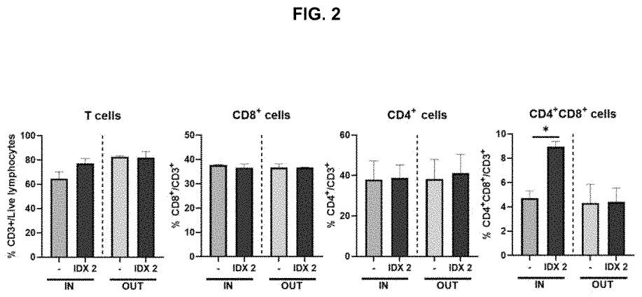

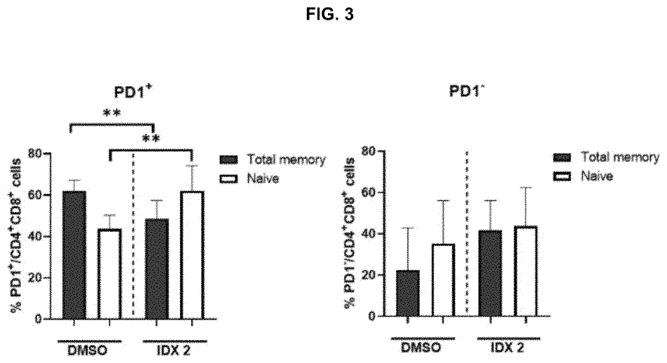

These findings support the use of idronoxil to induce immunomodulatory responses, and in combination with conventional chemotherapies, enhance antitumor immunity. By comparing CD3⁺ cell populations IN and OUT of the spheroids, the inventors observed a significant increase in double-positive (DP) T cells in the tumor structure upon idronoxil treatment (FIG. 2). Additionally, tumor-infiltrating DP T cells displayed a pronounced reduction in CD62L, but not CCR7, expression compared to control, suggesting that DP T cells could have a particular advantage toward spheroid infiltration following idronoxil treatment. Through gating analysis on CD3/CD4/CD8⁺ T cells following idronoxil-treated coculture, infiltrating DP T cells showed decreased proportions of CD45RO⁺ CD27⁺ memory cells and increased proportions for CD45RO⁻ CD27⁺ naïve cells, as compared to control. Finally, additionally to the above findings the inventors detected a significant reduction in PD1⁺ expression in total infiltrating memory cells compared to DMSO controls (mean 48.57±5.117 vs. mean 62.06±3.066; p=0.0086; FIG. 3). In contrast, significant up-regulation in PD1⁺ expression in naïve infiltrated cells upon idronoxil treatment as compared to DMSO (mean 62.10±7.049 vs. mean 43.57±3.870; p=0.0017) was found. These results indicate that DP memory T cells are prone to infiltrate spheroids with a weak exhaustion profile upon idronoxil treatment.

Generation of Tumor Cell Spheroids

Spheroids were generated using the liquid overlay technique. Therefore, a 96 well cell culture plate (Greiner) was precoated with 1.5% (w/v) agarose. For this, 0.75 g of agarose was diluted in 50 ml PBS and boiled for 15 min in a pressure cooker at full pressure, followed by another 10 min at lowest pressure level. 50 μl of agarose solution was added to each well of the 96 well plate and allowed to cool down at RT (under a cell culture hood). Tumor cells were trypsinized and adjusted to a cell suspension of 25.000 cells/ml in medium (RPMI 1640, 10% FCS, 1% penicillin/streptomycin). The outer wells of the 96 well plates were filled with PBS, and the remaining wells were filled with 200 μl cell suspension. Cell aggregation was triggered by centrifugation of the plate at 500× g, 5 min, RT. Plates were then maintained for 5 days in an incubator (37° C., humidified atmosphere). Medium was changed every two days by careful aspiration. Spheroid size was acquired with a Carl Zeiss Axiovert microscope and diameters were determined using AxioVision 40 software.

PMBC Isolation

PBMCs were isolated from buffy coats (DRK-Blutspendedients Baden-Würtemberg-Hessen, Institut für Transfusionsmedizin and Immunhämatologie, Frankfurt am Main, Germany) using Bicoll-Hypaque gradients. Two 50 ml Leukosep® tubes (Greiner) per buffy coat were filled with 15 ml lymphocyte separation medium (Sigma Aldrich) and centrifuged at 1000×g, 1 min, RT, to place the solution below the membrane. Afterwards 30 ml human blood from buffy coats was added, tubes were filled up to 50 ml with PBS/2 mM EDTA solution, and centrifuged at 500×g, 45 min, RT without break. After density gradient centrifugation, the intermediate white layer composed of mononuclear cells was transferred into a fresh sterile 50 ml tube and washed twice with PBS/2 mM EDTA. After RBC lysis (RBC lysis buffer: 135 mM NH₄Cl, 10 mM NaHCO₃, 0.1 mM EDTA; for 4 min at RT, stop with PBS) cells were diluted in RPMI 1640 medium (+10% FCS, 1% penicillin/streptomycin) at 2×10⁶ cells/ml.

Spheroid PBMC Co-Culture and Analysis

PBMCs were activated with 25 μl/ml CD3/CD28 T cell activator cocktail (StemCell Technologies), and 50.000 pre-activated PBMCs/well were added to spheroids. Afterwards, co-cultures were stimulated with 1 μM or 10 μM idronoxil or DMSO for 3 days. After three days, samples were either harvested for downstream analysis or treated again with 1 μM or 10 μM idronoxil or DMSO±10 μg/ml anti-PD-1 antibody (BioXCell) or the isotype control for another 3 days. Spheroid size was acquired with a Carl Zeiss Axiovert microscope and diameters were determined using AxioVision 40 software. Spheroids were harvested by transferring 5 spheroids of each group into FACS tubes (BD Biosciences), followed by centrifugation (500× g, 5 min, 4° C.). After removing the supernatant, 100 μl Accutase (Sigma Aldrich) was added to the spheroids, followed by 15 min incubation at 37° C. and generation of single cell suspensions by shearing the cell suspension repeatedly trough pipetting using 100 μl filter pipette tips. After centrifugation (500×g, 5 min, 4° C.) and removal of supernatant, cells were blocked with 80 μl of 0.5% BSA/PBS and 2 μl FcR-blocking reagent (Miltenyi Biotec) for 15 min on ice. Thereafter, cells were stained with an antibody mix consisting of anti-human CD4 PE-CF594, anti-human CD8 APC-H7, anti-human TCRab FITC, anti-human CD33 BV510, anti-human CD45 AF700, anti-human CD279 APC, and anti-human CD274 BV421 (each from BD Biosciences) antibodies for 20 min on nice in the dark. After washing with 500 μl FACS Flow (BD Biosciences), cells were resuspended in 300 μl FACS Flow, 20 μL absolute count standard (Bangs Laboratory) were added to each sample, and samples were acquired using an LSR II/Fortessa flow cytometer (BD Biosciences). All Antibodies were previously titrated to determine optimal concentrations. Antibody-capturing CompBeads (BD Biosciences) were used to create the multi-color panel compensation matrix. For gating, fluorescence minus one (FMO) controls and/or isotype controls were used. Instrument calibration was controlled and adjusted daily using Cytometer Setup and Tracking (CST) beads. For analysis of FACS data the Flow Jo V10 was used. Statistics were done using GraphPad Prism V8.

Tumour 3D spheroids of MCF-7 mammary carcinoma and A549 lung adenocarcinoma cells (starting at 10,000 cells) were grown for 5 days. On day 5, the cells were infiltrated with PBMCs (50.000, pre-activated with anti CD3/CD28 beads) and treated with DMSO or idronoxil (1 μM or 10 μM). After a further 3 days the cells were given a second treatment with DMSO or idronoxil (1 μM or 10 μM) alone or idronoxil in combination with anti-PD1 antibody. The Spheriod morphology was examined by microscopy and the cell number cell numbers (tumor cells and immune cell subsets) and PDL1/PD1 expression were measured using FACS with a FACS panel of CD45, CD33 (myeloid cells), CD3, CD4, CD8, PD1, PDL1.

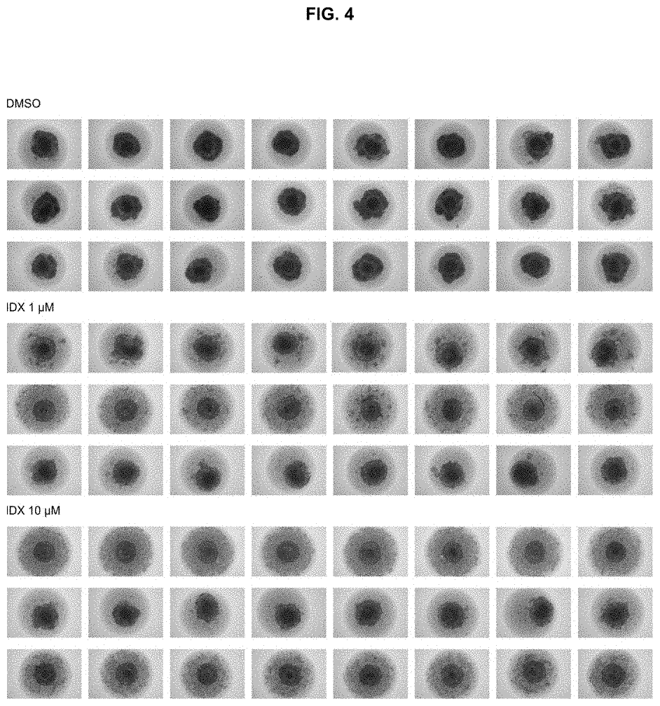

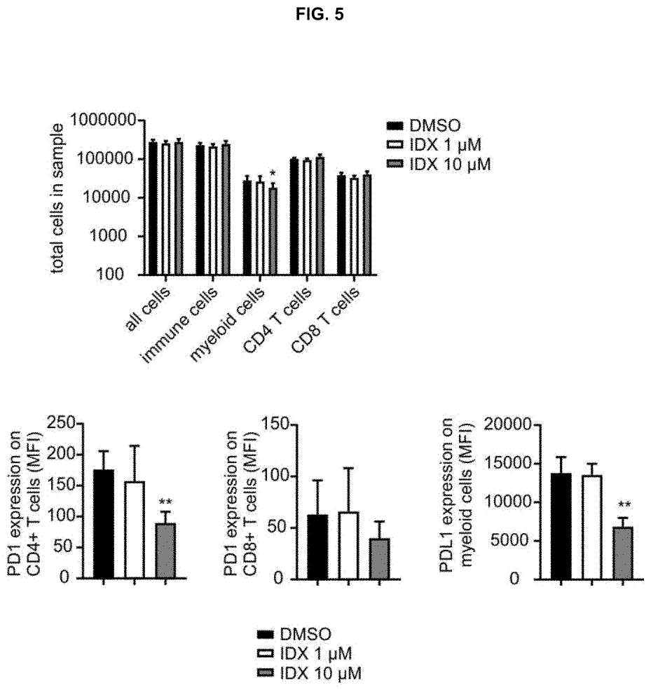

Idronoxil treatment in MCF-7 spheroids on day 3 of the treatment protocol reduced 3D spheroid size and induced or maintained immune activation (cluster formation). The spheroids appeared as clusters of tumor cells surrounded by corona of immune cells. This is shown in FIG. 4. FACS measurements reveal that idronoxil 10 μM treatment on MCF-7 spheroids on day 3 reduces the number of myeloid cells and reduces the expression of PD1 on CD4+ T cells and PDL1 on myeloid cells (FIG. 5).

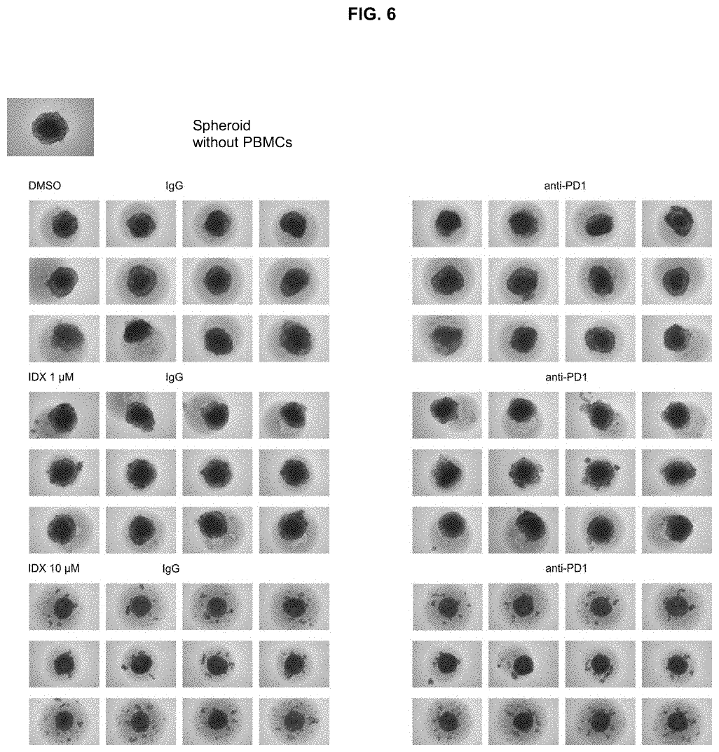

On day 6 of the treatment protocol, MCF-7 cells treated with idronoxil at 10 μM had reduced 3D spheroid size. The spheroids displayed cluster formation indicated that immune function of the cells was maintained. The spheroids appeared as clusters of tumor cells surrounded by corona of immune cells (FIG. 6).

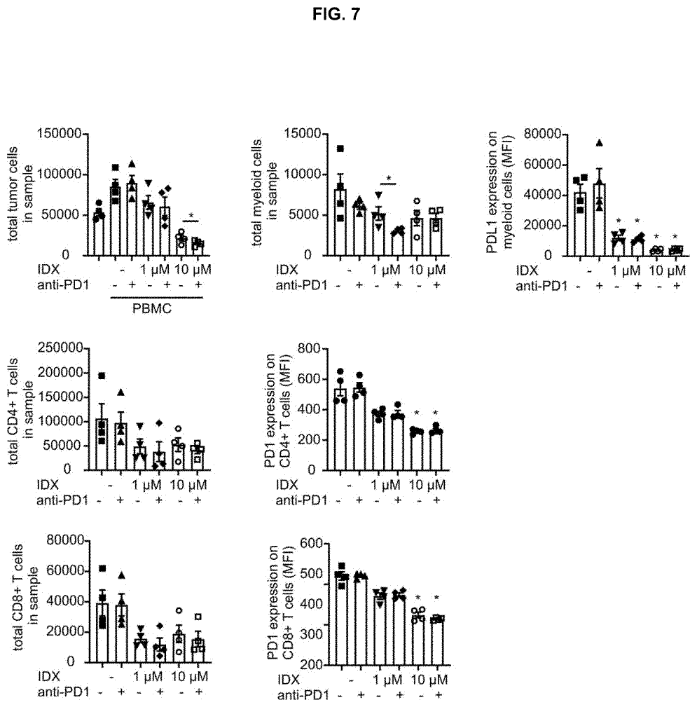

The FACS measurements (FIG. 7) on day 6 of the treatment protocol for the MCF-7 spheroids demonstrate that idronoxil at 10 μM treatment reduces the number of tumor cells. This is enhanced by anti-PD1 treatment. Idronoxil 1 μM+anti-PD1 reduces the number of myeloid cells and idronoxil in general strongly reduces PDL1 expression by myeloid cells. Idronoxil, especially at 10 μM, reduces the expression of PD1 on both, CD4+ and CD8+ T cells.

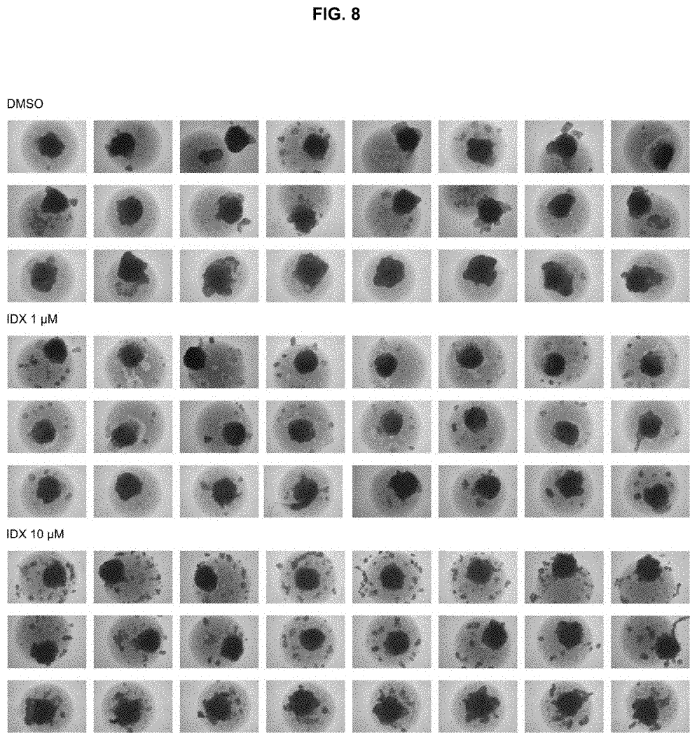

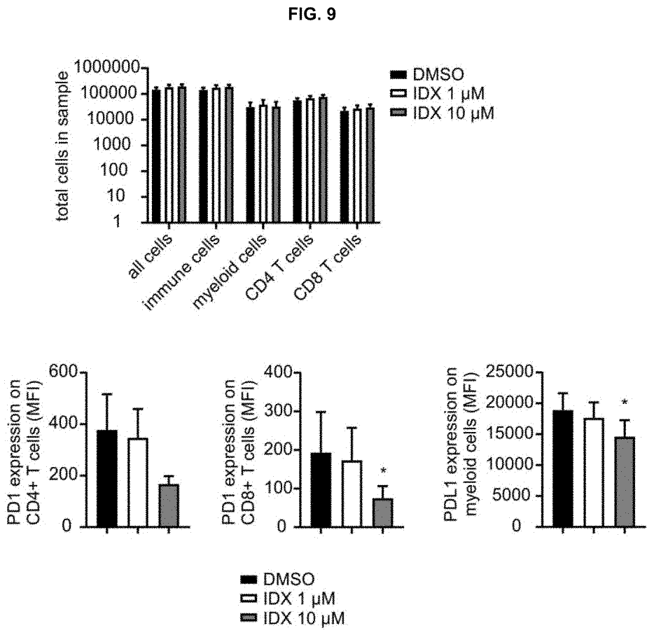

Idronoxil evaluation of the A549 spheroids on day 3 of the protocol (FIG. 8) showed that idronoxil at 10 μM reduces 3D spheroid size and that the idronoxil at 10 μM treatment induces or maintains immune activation (cluster formation). The spheroids appeared as clusters of tumor cells surrounded by a corona of immune cells. The FACS measurements (FIG. 9) showed that idronoxil 10 μM treatment reduced the expression of PD1 on CD8+ T cells and PDL1 on myeloid cells.

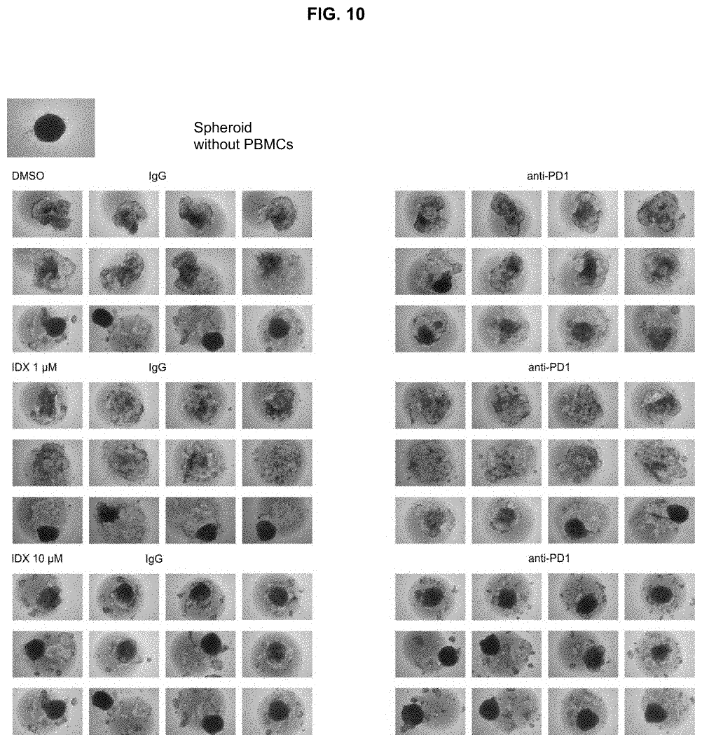

On day 6 of the treatment protocol, A549 spheroids had a different appearance, appearing as loose clusters of tumor cells (FIG. 10); invasion into agarose, still surrounded by corona of immune cells. Idronoxil at 1 μM treatment reduced 3D spheroid size, and treatment with idronoxil at 10 μM reduced size and invasion of the cells (evidenced by stabilised spheric appearance). Idronoxil at 10 μM maintains immune activation (cluster formation). There was no obvious alteration in appearance between cells receiving the idronoxil treatment or the idronoxil+anti-PDI treatment.

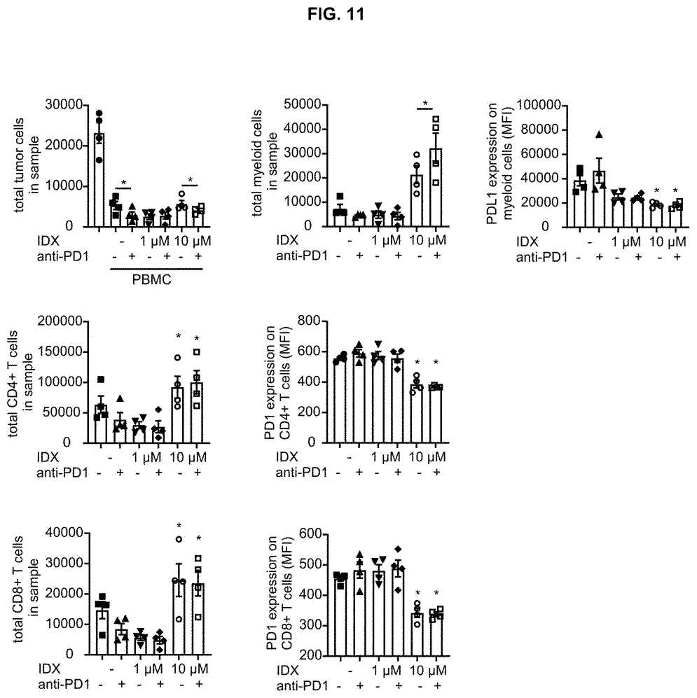

The FACS measurements of the A549 spheroids on day 6 of the treatment protocol (FIG. 11) showed that PBMCs in general reduce the number of tumor cells, which was enhanced by anti-PD1 treatment in control conditions, or upon treatment 10 μM. Idronoxil 10 μM increased immune cell infiltrates and myeloid cells were further enhanced by anti-PD1 treatment compared to idronoxil treatment alone. Idronoxil treatment at 10 μM, reduces the expression of PD1 on both, CD4+ and CD8+ T cells, as well as PDL1 on myeloid cells.

In summary it was found that idronoxil reduces PD1 and PDL1 expression, thereby being immunogenic by itself. The anti-PD1 treatment increases tumor killing upon 10 μM idronoxil treatment; in A549 spheroids idronoxil 10 μM prevents invasion and the immune infiltrate is modulated by idronoxil, but the direction depends on the tumor properties.

### CLINICAL EXAMPLES

### Example 4

Many clinicians treat patients with checkpoint inhibitors or 10 drugs despite apparent progressive disease. These patients follow three subsequent patterns:
Hyper-progression i.e. very rapid progression. This is more common the pseudo-progression and is associated with age>70
Pseudoprogression i.e. despite initially demonstrating tumor growth of greater than 20% they subsequently go on to develop a good response (partial or complete). These patients tend to be younger and have a lower overall tumor burden.
Other progressors—they sort of continue to meander upwards slowly over time.

In those patients that do not progress early (approx. 12 weeks following treatment initiation):
Most develop partial response at some point:
   Most of these do so within 24 weeks
   A smaller group meander along and either stay stable or tip over (modestly) in partial response after a year or more
   Very few develop complete response

A clear overall survival benefit difference is apparent and favouring those without progressive disease within 12 weeks from baseline compared with those that do demonstrate progressive disease within 12 weeks.

### Example 5

An individual presents with metastatic castration resistant prostate cancer. The tumor exhibits an immune marker profile indicating poor responsiveness to anti-PD-1 antibody therapy. The individual has not been prior treated with an 10 drug. The individual is given 800 mg daily dosage of idronoxil for a period of 7 days to establish a plasma concentration of 350 ng/mL of idronoxil. The individual is then administered with an anti-PD-1 antibody according to the product information. The dosage cycle is repeated twice and the individual is assessed for response to anti PD-1 therapy.

### Example 6

An individual presents with advanced melanoma, having been assessed at 12 weeks from first administration of an anti-CTLA-4 antibody as having stable disease and not having achieved a partial response. The individual is given 800 mg daily dosage of idronoxil for a period of 7 days to establish a plasma concentration of 350 ng/mL of idronoxil. The individual is then administered with an anti-CTLA-4 antibody according to the product information. The dosage cycle is repeated twice and the individual is assessed for response to anti-CTLA-4 therapy.

### Example 7

An individual presents with a solid tumor, having been assessed at least 2 weeks from first administration of a PD-1 inhibitor antibody, preferably nivolumab, as having no response or a partial response to the PD-1 inhibitor antibody.

Alternatively an individual presents with or is suspected of having a tumor assessed as being likely to develop a partial response, or no response to a PD-1 antibody, preferably nivolumab, wherein the individual has not been administered the PD-1 antibody.

In one embodiment, the individual is given 1200 mg daily dosage of idronoxil for a period of 10 days. The individual is administered with 240 mg nivolumab intravenously on day 2. On days 11 to 14 the individual does not receive treatment of idronoxil or nivolumab. The treatment cycle is 2 weekly. The individual is assessed for response to nivolumab therapy.

In another embodiment, the individual is given 1200 mg daily dosage of idronoxil for a period of 10 days. The individual is administered with 480 mg nivolumab intravenously on day 2. On days 11 to 28 the individual does not receive treatment of idronoxil or nivolumab. The treatment cycle is 4 weekly. The individual is assessed for response to nivolumab therapy.

1. A method for improving a response in an individual to immuno-oncology therapy for cancer, wherein the immuno-oncology therapy is a checkpoint inhibitor therapy, comprising the step of administering a compound of Formula 1: 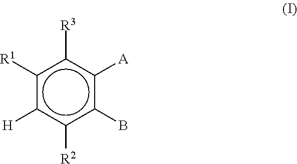
wherein
R¹ is H, C₁₋₁₀ alkyl, C₁₋₁₀ haloalkyl, OH, OR^{A} or OC(O)R^{A} where R^{A} is C₁₋₁₀ alkyl, C₁₋₁₀ haloalkyl or an amino acid;
R² is H, OH, or R^{B} where R^{B} is an amino acid or COR^{A} where R^{A} is as previously defined;
R³ is H, halo or C₁₋₁₀ alkyl;
A and B together with the atoms between them form a six membered ring selected from the group 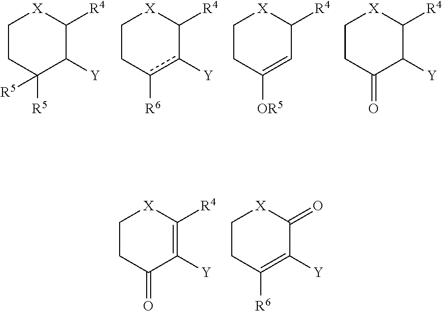
wherein
R⁴ is H, COR^{D} where R^{D} is H, OH, C₁₋₁₀ alkyl or an amino acid, CO₂R^{C} where R^{C} is C₁₋₁₀ alkyl, COR^{E} where R^{E} is H, C₁₋₁₀ alkyl or an amino acid, COOH, COR^{C} where R^{C} is as previously defined, or CONHR^{E} where R^{E} is as previously defined;
R⁵ is H, CO₂R^{C} where R^{C} is as previously defined, or COR^{C}OR^{E} where R^{C} and R^{E} are as previously defined, and where the two R⁵ groups are attached to the same group they are the same or different;
R⁶ is H, CO₂R^{C} where R^{C} is as previously defined, COR^{C}OR^{E} where R^{C} and R^{E} are as previously defined, substituted or unsubstituted aryl or substituted or unsubstituted heteroaryl;
X is O, N or S;
Y is selected from the group 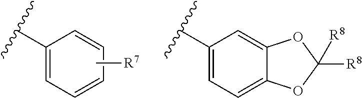
wherein
R⁷ is H, C₁₋₁₀ alkyl, C₁₋₁₀ haloalkyl, halo, OR^{F} where R^{F} is H, C₁₋₁₀ alkyl, C₁₋₁₀ haloalkyl, or OC(O)R^{A} where R^{A} is as previously defined;
R⁸ is H or halo; and
“ [symbol] ” represents either a single bond or a double bond;
to an individual in whom an improved response to immuno-oncology therapy is required, thereby improving a response to immuno-oncology therapy in the individual.

2. (canceled)

3. The method of claim 1, comprising the further step of administering the immuno-oncology therapy to the individual to treat the individual for cancer.

4. The method of claim 1, wherein the individual is one who has been prior administered with the immuno-oncology therapy and in whom a partial response to the immuno-oncology therapy has developed.

5. The method of claim 1, wherein the individual is one who has been prior administered with the immuno-oncology therapy and in whom no response to the immuno-oncology therapy has developed.

6. The method of claim 4, wherein the individual develops an improved response in the form of a complete response to immuno-oncology therapy, after the administration of a compound of Formula 1.

7. The method of claim 5, wherein the individual develops an improved response in the form of a partial response, or a complete response to immuno-oncology therapy after the administration of a compound of Formula 1.

8. The method of claim 1, wherein the individual has not been administered with the immuno-oncology therapy prior to the administration of a compound of Formula 1.

9. The method of claim 1, wherein the individual has been assessed as being likely to develop a partial response, or no response to immuno-oncology therapy for cancer, prior to administration with a compound of Formula 1.

10. The method of claim 1, wherein the compound of Formula 1 is idronoxil.

11. The method of claim 1, wherein the compound of Formula 1 is provided in the individual to establish a plasma concentration of 40 ng/mL to 400 μg/mL in the individual.

12. The method of claim 1, wherein the compound of Formula 1 is provided in the individual to establish a plasma concentration of 40 ng/mL to 400 μg/mL in the individual for a period of at least one half life of the immuno-oncology therapy.

13. The method of claim 1, wherein the immuno-oncology is administered to the individual at the time that a plasma concentration of a compound of Formula 1, of about 40 ng/mL to 400 μg/mL has been established in the individual.

14. The method of claim 1, wherein the immuno-oncology therapy and a compound of Formula 1 are administered to the individual at the same time.

15. (canceled)

16. The method of claim 1, wherein the individual is not treated with, or has not been treated with radiotherapy or chemotherapy for treatment of the cancer.

17-18. (canceled)

19. The method of claim 1, wherein the checkpoint inhibitor therapy is a CTLA-4 inhibitor.

20. The method of claim 1, wherein the checkpoint inhibitor therapy is a PD-1 inhibitor.

21. The method of claim 1, wherein the checkpoint inhibitor therapy is a PD-L1 inhibitor.
